(19) United States
(12) Patent Application Publication
(10) Pub. No.: US 2021/0264315 A1
(43) Pub. Date: Aug. 26, 2021

(54) SELECTION ASSISTANCE DEVICE, SELECTION ASSISTANCE METHOD, DATA STRUCTURE, LEARNED MODEL, AND PROGRAM

(71) Applicant: Nippon Telegraph and Telephone Corporation, Tokyo (JP)

(72) Inventors: Manabu Yoshida, Tokyo (JP); Atsuhiko Maeda, Tokyo (JP); Ippei Shake, Tokyo (JP)

(21) Appl. No.: 17/260,409

(22) PCT Filed: Jul. 1, 2019
(86) PCT No.: PCT/JP2019/026096

§ 371 Date: Jan. 14, 2021

(30) Foreign Application Priority Data
Jul. 17, 2018 (JP) 2018-134132

(51) Int. Cl. G06N 20/00; G06Q 10/06

(52) U.S. Cl. CPC G06N 20/00; G06Q 50/26; G06Q 10/0639

(57) ABSTRACT

A technology for allowing a facility highly likely to accept a request from a user to be more efficiently selected is provided. A selection assistance apparatus for assisting in selecting an acceptance destination facility in response to a request from a user acquires acceptance performance data in which information indicating success or failure of acceptance for a past acceptance request in each of a plurality of candidate facilities is associated with attribute information relevant to the past request, calculates a past probability of acceptance according to the attribute information in each of the plurality of candidate facilities, and generates a prediction model used for prediction of a likelihood of acceptance for a newly generated acceptance request according to attribute information relevant to the newly generated acceptance request for each of the plurality of candidate facilities, the prediction model indicating a relationship between information indicating success or failure of the acceptance and the attribute information.

### TECHNICAL FIELD

One aspect of the present invention relates to a selection assistance apparatus, a selection assistance method, a data structure, a learned model and a program that assist in selecting an acceptance destination facility in response to a request from a user.

### BACKGROUND ART

When an acceptance destination facility is selected in response to a request from a user, it may be difficult to search for a facility that accepts the request. For example, a case in which there is a request for rescue transport and a hospital that is a transport destination is searched for is conceivable.

One known problem in transporting a patient to a hospital using a rescue vehicle in response to a request for rescue transport is that it takes time to identify a hospital that is able to accept the patient. In particular, when acceptance is rejected by a hospital that is a destination to which the transport request has been output and a hospital that is a transport destination must be selected again, the time required for transport may be greatly increased.

To solve this problem, an apparatus that displays a list of medical institutions that has past acceptance performance on a terminal owned by rescue personnel based on severity and symptoms of a patient (see Patent Literature 1, for example), a system that identifies a hospital having a hospital visit record as a transport destination by enabling a patient to utilize history data indicating past hospital visits when the patient desires rescue transport (see Patent Literature 2, for example), and a system that identifies a hospital that is a transport destination in a short time by setting a candidate group of hospitals to which a rescue acceptance request is preferentially made in advance and broadcasting to the hospitals a mail for inquiring about whether or not the hospitals can accept the request (see Patent Literature 3, for example), and the like have been reported.

### CITATION LIST

### Patent Literature

Patent Literature 1: Japanese Unexamined Patent Application Publication No. 2016-35699

Patent Literature 2: Japanese Unexamined Patent Application Publication No. 2014-219854

Patent Literature 3: Japanese Unexamined Patent Application Publication No. 2007-128245

### SUMMARY OF THE INVENTION

### Technical Problem

However, in the technology described in Patent Literature 1, because a plurality of hospital candidates that are transport request destinations are displayed, it takes time to identify an acceptable hospital. In the technology described in Patent Literature 2, because only a hospital having a hospital visit record is selected, the hospital is likely to be unable to handle a patient according to a degree of severity or symptom of the patient. In the technology described in Patent Literature 3, the assumption that a rescue vehicle and each hospital are connected to a communication network and can exchange mails via a rescue assistance server is not necessarily true.

The present invention has been made in light of the above circumstances, and an object of the present invention is to provide a technology for enabling a facility highly likely to accept a request from a user to be predicted.

### Means for Solving the Problem

in order to solve the above problem, a first aspect of the present invention is directed to a selection assistance apparatus for assisting in selecting an acceptance destination facility in response to a request from a user, the selection assistance apparatus including: an acceptance performance data acquisition unit configured to acquire acceptance performance data in which information indicating success or failure of acceptance for a past acceptance request in each of a plurality of candidate facilities is associated with attribute information relevant to the past acceptance request; a past probability calculation unit configured to calculate a past probability of acceptance according to the attribute information in each of the plurality of candidate facilities based on the acquired acceptance performance data and a learning unit configured to generate a prediction model for predicting a likelihood of acceptance for a newly generated acceptance request according to attribute information relevant to the newly generated acceptance request for each of the plurality of candidate facilities based on the acceptance performance data and the calculated past probability, the prediction model indicating a relationship between information indicating success or failure of the acceptance and the attribute information.

A second aspect of the present invention is directed to the selection assistance apparatus according to the first aspect, further including: an acceptance likelihood prediction unit configured to predict a likelihood of acceptance of the newly generated acceptance request based on the generated prediction model and attribute information relevant to the newly generated acceptance request for each of the plurality of candidate facilities; and an output unit configured to output a result of the prediction of the acceptance likelihood prediction unit.

A third aspect of the present invention is directed to the selection assistance apparatus according to the second aspect, wherein the acceptance likelihood prediction unit further calculates a score value indicating a level of the likelihood of acceptance; and the output unit sorts and outputs the calculated score values.

A fourth aspect of the present invention is directed to the selection assistance apparatus according to the first aspect, wherein the learning unit generates the prediction model for each feature type focusing on at least one of a plurality of features extracted from the attribute information.

A fifth aspect of the present invention is directed to the selection assistance apparatus according to any one of the first to fourth aspects, wherein the past probability calculation unit calculates a past probability in each of the plurality of candidate facilities under conditions corresponding to each of a plurality of features extracted from the attribute information relevant to the past acceptance request, and the learning unit generates the prediction model using information indicating success or failure of the acceptance as an objective variable, and at least one of the plurality of features and the past probability as an explanatory variable.

### Effects of the Invention

According to the first aspect of the present invention, the past probability of acceptance according to the attribute information in each of the candidate facilities is calculated based on the acceptance performance data in which the information indicating success or failure of the acceptance for the past acceptance request in the candidate facilities is associated with the attribute information relevant to the acceptance request. The prediction model indicating the relationship between the information indicating success or failure of the acceptance and the attribute information is generated based on the acceptance performance data and the calculated past probability. Using the prediction model generated in this manner, it is possible to predict the likelihood of acceptance of each facility for the new acceptance request based on the attribute information relevant to the new acceptance request when the new acceptance request is generated. Because the prediction model is generated based on past statistical data, it is possible to realize a more reliable prediction. Further, because the prediction model considers the attribute information, the prediction model can also be useful for analysis of how the attribute information contributes to the success or failure of acceptance.

According to the second aspect of the present invention, the likelihood of the acceptance for each candidate facility for the newly generated acceptance request is predicted based on the attribute information relevant to the newly generated acceptance request, using the prediction model generated in the first aspect, and a prediction result is output. This allows the user to obtain a highly reliable prediction result considering the attribute information for a likelihood that the newly generated acceptance request will be accepted by the facility. The user can determine, for example, a candidate facility that is most likely to accept the newly generated acceptance request based on the output prediction result, and send a request for acceptance to the facility. Alternatively, the user can convert the prediction result into a numerical value to perform various computation processes.

According to the third aspect of the present invention, the acceptance likelihood prediction unit further calculates the score value indicating the level of the likelihood of the acceptance for each candidate facility for the newly generated acceptance request. This facilitates a computation process based on the score value and allows the prediction result to be utilized in various ways. Further, because the output unit sorts and outputs the calculated score values, it is possible to output the score value in a format that is easy for the user to use. Further, it is possible to curb a processing load of the apparatus by selecting the output according to the score value. A user can find a candidate facility having a high score value, thereby easily identifying a facility highly likely to accept the request.

According to the fourth aspect of the present invention, the learning unit generates the prediction model for each of types of features, focusing on at least one of the plurality of features extracted from the attribute information. This produces a more accurate prediction model considering types of features extracted from the attribute information.

According to the fifth aspect of the present invention, the past probability for the past acceptance request in each of the candidate facilities is calculated under conditions corresponding to each of the plurality of features extracted from the attribute information relevant to the acceptance request, and the prediction model is generated using the information indicating success or failure of the acceptance as an objective variable, and the at least one of the plurality of features and the past probability as an explanatory variable. This allows a precise prediction model considering how each of the features extracted from the attribute information contributes to success or failure of the acceptance to be generated. Using this prediction model, it is possible to realize a highly accurate prediction that further satisfies a condition depending on each of the features extracted from the attribute information relevant to the newly generated acceptance request.

That is, according to each aspect of the present invention, it is possible to provide a technology for enabling prediction of a facility that is highly likely to accept an acceptance request from a user.

### BRIEF DESCRIPTION OF DRAWINGS

FIG. 1 is a block diagram illustrating a functional configuration of a selection assistance apparatus according to an embodiment of the present invention.

FIG. 2 is a flow diagram illustrating an example of a processing procedure and processing content of a past probability calculation process in the selection assistance apparatus illustrated in FIG. 1.

FIG. 3 is a flow diagram illustrating an example of a processing procedure and processing content of a prediction model generation process in the selection assistance apparatus illustrated in FIG. 1.

FIG. 4 is a flow diagram illustrating an example of a processing procedure and processing content of a score calculation data acquisition process in the selection assistance apparatus illustrated in FIG. 1.

FIG. 5 is a flow diagram illustrating an example of a processing procedure and processing content of a score calculation process in the selection assistance apparatus illustrated in FIG. 1.

FIG. 6 is a diagram illustrating an example of performance data D1 acquired by the selection assistance apparatus illustrated in FIG. 1.

FIG. 7 is a diagram illustrating an example of past probability data D2 calculated by the selection assistance apparatus illustrated in FIG. 1.

FIG. 8 is a diagram illustrating an example of prediction model generation data D3 acquired by the selection assistance apparatus illustrated in FIG. 1.

FIG. 9 is a diagram illustrating an example of prediction data D4 acquired by the selection assistance apparatus illustrated in FIG. 1.

FIG. 10 is a diagram illustrating an example of score calculation data D5 acquired by the selection assistance apparatus illustrated in FIG. 1.

FIG. 11 is a diagram illustrating an example of a coefficient vector W acquired by the selection assistance apparatus illustrated in FIG. 1.

FIG. 12 is a diagram illustrating an example of output data including a score value calculated by the selection assistance apparatus illustrated in FIG. 1.

FIG. 13A is a diagram illustrating a second example of the prediction model generation data D3 acquired by the selection assistance apparatus illustrated in FIG. 1.

FIG. 13B is a diagram illustrating a third example of the prediction model generation data D3 acquired by the selection assistance apparatus illustrated in FIG. 1.

FIG. 14A is a diagram illustrating a second example of the coefficient vector W acquired by the selection assistance apparatus illustrated in FIG. 1.

FIG. 14B is a diagram illustrating a third example of the coefficient vector W acquired by the selection assistance apparatus illustrated in FIG. 1.

### DESCRIPTION OF EMBODIMENTS

Hereinafter, an embodiment of the present disclosure will be described in detail with reference to the drawings.

### Embodiment

### Configuration

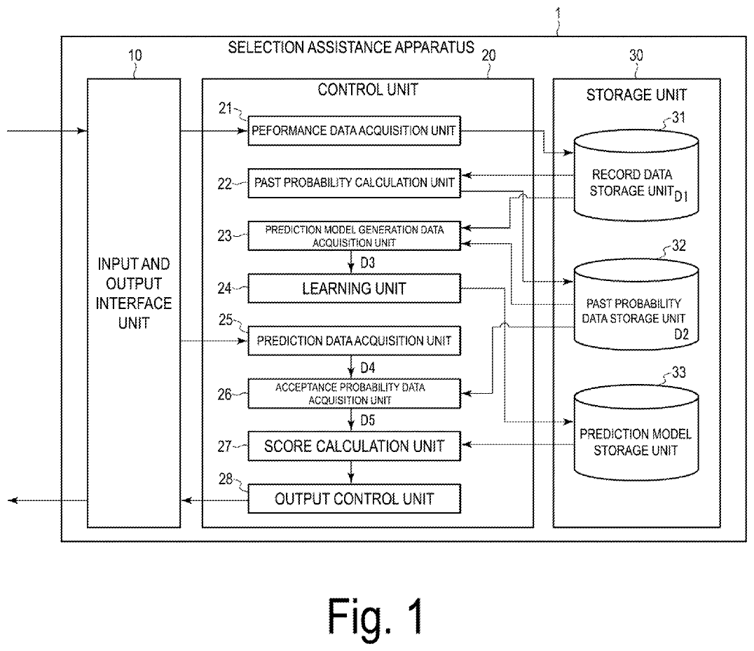

FIG. 1 is a block diagram illustrating a functional configuration of a selection assistance apparatus 1 according to an embodiment of the present invention. Hereinafter, a case in which, when there is a rescue transport request as an acceptance request from a user, the user or an operator (for example, rescue personnel or an operator of a service center), or the like selects a facility that is a transport destination and makes a transport request to the facility will be described by way of example. Here, the acceptance request is not limited to only such a transport request, and an acceptance destination facility of the request is not limited to a medical institution.

The selection assistance apparatus 1 according to an embodiment includes, for example, a personal computer or a server apparatus. The selection assistance apparatus 1 generates a prediction model that is used for prediction of a likelihood that a transport destination candidate facility such as a hospital will accept the transport request when there is a patient needing rescue transport, to assist a user or the like in selecting the facility that is a transport destination. The selection assistance apparatus 1 includes, as hardware, an input and output interface unit 10, a control unit 20, and a storage unit 30.

The input and output interface unit 10 includes, for example, one or more wired or wireless communication interface units. The input and output interface unit 10 inputs various types of data input by an input device (not illustrated) including, for example, a keyboard or a mouse to the control unit 20. Further, the input and output interface unit 10 causes display data output from the control unit 20 to be displayed on a display device (not illustrated) such as a liquid crystal display. The input and output interface unit 10 enables information to be transmitted to and received from an external server, an external database, or the like via a communication network.

For the storage unit 30, a nonvolatile memory on which writing and reading can be performed at any time, such as a hard disc drive (HDD) or a solid state drive (SSD), for example, is used as a storage medium. Further, the storage unit 30 includes a performance data storage unit 31, a past probability data storage unit 32, and a prediction model storage unit 33, as storage regions required for realization of the embodiment.

The performance data storage unit 31 stores acceptance performance data D1 including information on a past acceptance request and information indicating whether or not the request is successfully accepted. The acceptance performance data D1 is performance data in which identification information of the facility that is a transport destination, acceptance result information indicating whether or not each facility has accepted a transport request, and attribute information relevant to the transport request are associated with each other. The attribute information is various types of information on the acceptance request from the user. For example, when the request from the user is a request for rescue transport, a date, a day of the week, a time period, weather, a symptom of a patient, a clinic practice area corresponding to the symptom of the patient, a complexion of the patient, a heart rate of the patient, and the like when there has been the request for rescue transport (each of which is also hereinafter referred to as a “feature extracted from the attribute information”) are included in the attribute information. Further, the acceptance performance data can include attribute information on the candidate facilities. For example, when the number of available beds, work information of a specialist, and the like associated with the identification information of the facility that is a transport destination can be acquired, the performance data D1 can include such information.

The past probability data storage unit 32 stores past probability data D2 including information indicating a probability (past probability) that each facility will accept a transport request, which is calculated based on the performance data D1.

The prediction model storage unit 33 stores a prediction model that is used for prediction of a likelihood that each candidate facility will accept an acceptance request, based on attribute information relevant to a newly generated acceptance request.

The control unit 20 includes a hardware processor such as a central processing unit (CPU), and a program memory, which are not illustrated. The control unit 20 includes a performance data acquisition unit 21, a past probability calculation unit 22, a prediction model generation data acquisition unit 23, a learning unit 24, a prediction data acquisition unit 25, a past probability data acquisition unit 26, a score calculation unit 27, and an output control unit 28 in order to execute a processing function in the embodiment. All of the processing functions of these units are implemented by causing the hardware processor to execute a program stored in the program memory. Note that these processing functions may be implemented not by using a program stored in the program memory, but by using a program provided through a network.

The performance data acquisition unit 21 acquires the performance data D1 regarding the past acceptance request from an input device, an external database, or the like (not illustrated) via the input and output interface unit 10, and stores the acquired performance data D1 in the performance data storage unit 31. The performance data D1 is, for example, performance data including information on a patient (for example, a symptom or vital data) and information on an environment (for example, a day of the week or a time period).

The past probability calculation unit 22 executes a process of reading data stored in the performance data storage unit 31 of the storage unit 30 and generating a data set D2 indicating a probability that a past request was accepted for each piece of attribute information or for each feature extracted from the attribute information. The past probability calculation unit 22 may calculate the probability from all pieces of past data, may calculate the probability from data for the past month, or may calculate both probabilities. Alternatively, the past probability calculation unit 22 may calculate the probability from data in any period of time including any past point in time. In one example, the past probability calculation unit 22 divides the performance data D1 for each facility (hospital), further divides the data in units of years and units of months for each facility, calculates the past probabilities for each clinic practice area and each day of the week, and acquires the data set D2. Thereafter, the past probability calculation unit 22 causes the acquired data set D2 to be stored in the past probability data storage unit 32 of the storage unit 30.

The prediction model generation data acquisition unit 23 performs a process of reading data stored in the performance data storage unit 31 and the past probability data storage unit 32 of the storage unit 30 and acquiring prediction model generation data D3, which is used to generate the prediction model. The prediction model generation data D3 will be further described below. The prediction model generation data acquisition unit 23 outputs the acquired prediction model generation data D3 to the learning unit 24.

The learning unit 24 executes a process of performing statistical analysis using the prediction model generation data D3. For example, the learning unit 24 executes a process of calculating a coefficient vector associated with a model for calculating a score value indicating likelihood of occurrence of the label (acceptable) from the feature vector by using information on a patient in the prediction model generation data D3 or a past probability of acceptance as a feature vector, and a value indicating success or failure of acceptance (acceptance rejection or acceptable) included in the data set as a correct answer label. The calculated coefficient vector is stored in the prediction model storage unit 33. The coefficient vector calculated according to the feature vector can be used for a prediction process as the prediction model. Hereinafter, a prediction model in which the coefficient vector has been determined (learned) is also referred to as a learned model.

When a new acceptance request is generated, for example, when there is a patient needing transport, the prediction data acquisition unit 25 acquires data indicating attribute information relevant to the transport request as the prediction data D4 and outputs the data to the past probability data acquisition unit 26.

The past probability data acquisition unit 26 reads past probability data satisfying conditions (for example, a clinic practice area corresponding to a symptom of a patient, or a day of the week) from the past probability data D2 stored in the past probability data storage unit 32 of the storage unit 30 based on the acquired prediction data D4, and outputs the past probability data together with the prediction data D4 as score calculation data D5.

The score calculation unit 27 calculates a score value indicating a likelihood that a transport request will be accepted when the transport request is made to a certain specific facility, using the score calculation data D5 output from the past probability data acquisition unit 26 and a pre-generated prediction model stored in the prediction model storage unit 33. In the embodiment, the score calculation unit 27 can calculate the score value using the past probability data as a feature vector and using the coefficient vector stored in the prediction model storage unit 33.

The output control unit 28 performs a process of creating output data based on the score values calculated by the score calculation unit 27 and outputting the data to a display device or an external terminal (not illustrated) via the input and output interface unit 10. For example, the output control unit 28 can create, as output data, a priority list obtained by assigning priorities to transport destination candidate facilities based on score values calculated for a plurality of transport destination candidate facilities. The output control unit 28 may create the calculated score value for each of the plurality of candidate facilities as the output data or may create the score values as output data in which candidate facilities other than the sorted upper-ranked candidate facilities are excluded.

Operation Next, an operation of the selection assistance apparatus 1 configured as described above will be described using several examples.

### First Example

### (1) Calculation of Past Probability

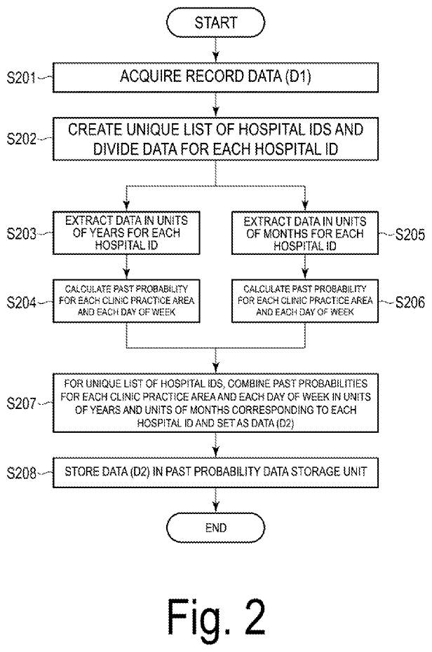

FIG. 2 is a flow diagram illustrating an example of a processing procedure and processing content of a past probability calculation process in the control unit 20 of the selection assistance apparatus 1 illustrated in FIG. 1. This process may be started at any timing and, for example, may be started automatically at certain time intervals or may be started using an operation of an operator as a trigger.

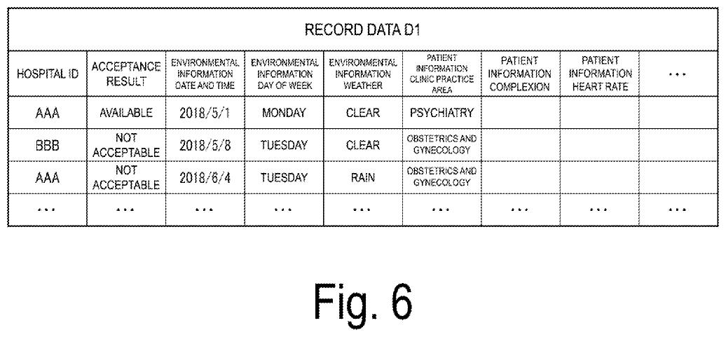

In step S201, the control unit 20 acquires performance data D1 according to past transport performance from an input device, an external database, or the like via the input and output interface unit 10 under the control of the performance data acquisition unit 21, and stores the performance data D1 in the performance data storage unit 31. For example, the control unit 20 can capture data input manually by the operator through an input device including a keyboard, a mouse, or the like as the performance data D1. Alternatively, the acquisition of the data may be executed through automatic collection using communication. FIG. 6 illustrates an example of the acquired performance data D1. At least a column of a hospital ID for identifying the facility that is a transport destination and an acceptance result column indicating a result of making the transport request to the hospital are included in the performance data D1. A date and time, a day of the week, and weather as environmental information, a clinic practice area, a complexion of a patient, and a heart rate of the patient as patient information, and the like may be included in the performance data D1. Further, when various types of attribute information associated with the hospital ID can be acquired, such information may be included in the performance data D1. As such attribute information, a wide variety of information such as a total number of beds, the number of available beds, work information of a specialist, and the number of doctors for each clinic practice area may be included in the performance data D1.

In step S202, the control unit 20 performs a process of reading the performance data D1 from the performance data storage unit 31, referring to the column of the hospital ID of the performance data D1, creating a unique list of hospital IDs, and dividing the performance data D1 for each hospital ID under control of the past probability calculation unit 22.

Subsequently, in step S203, the past probability calculation unit 22 extracts data in units of years for each piece of data divided for each hospital ID.

Then, in step S204, the past probability calculation unit 22 calculates, for each hospital, past probabilities for each clinic practice area and each day of the week based on the data extracted in units of years. The past probability is a ratio between the number of times the transport request has been made, that is, the so-called number of records of data, as a denominator and the number of records that are “acceptable” in the acceptance result column of the data as a numerator, and is calculated to range from 0 to 1.

Similarly, in step S205, the past probability calculation unit 22 extracts data in units of months for each piece of data divided for each hospital ID.

Then, in step S206, the past probability calculation unit 22 calculates the past probabilities for each clinic practice area and each day of the week based on the data extracted in units of months. The past probability is calculated to range from 0 to 1, as in step S204. Steps S203 to S204 and steps S205 to S206 may be executed concurrently or may be executed sequentially.

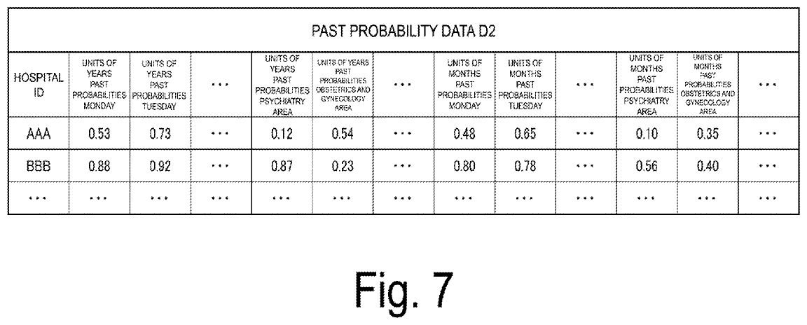

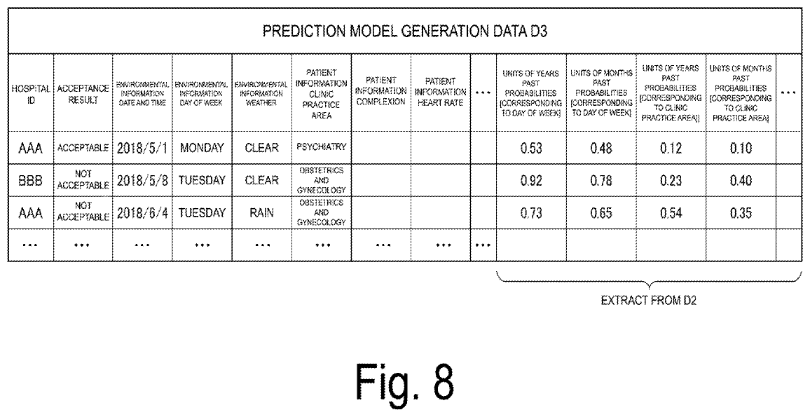

In step S207, the past probability calculation unit 22 combines the calculated past probabilities with a corresponding hospital ID in the unique list of the hospital IDs and sets the resultant data as the past probability data D2. FIG. 7 illustrates an example of the past probability data D2. A hospital ID as identification information of a candidate facility, and a probability of Monday calculated in units of years, a probability of Tuesday calculated in units of years, a probability of a psychiatry area calculated in units of years, a probability of an obstetrics and gynecology area calculated in units of years, a probability of Monday calculated in units of months, a probability of Tuesday calculated in units of months, a probability of a psychiatry area calculated in units of months, a probability of the obstetrics and gynecology area calculated in units of months, as past probability of acceptance of each hospital, for example, are included in the past probability data D2. The probabilities included in the past probability data D2 are not limited to the units of year and the units of months, and the past probability calculation unit 22 may calculate the past probability for any time interval, such as in units of quarters, units of weeks, and units of days, and constitute the past probability data D2.

In step S208, the past probability calculation unit 22 stores the acquired past probability data D2 in the past probability data storage unit 32.

### (2) Generation of Prediction Model (Calculation of Coefficient Vector)

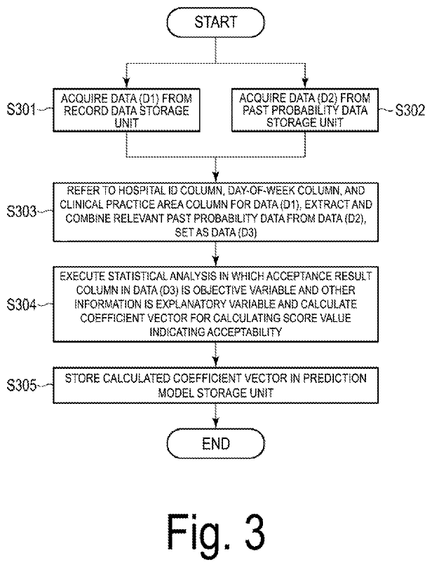

FIG. 3 is a flow diagram illustrating an example of a generation processing procedure and processing content of a prediction model in the control unit 20 of the selection assistance apparatus 1 illustrated in FIG. 1. In the embodiment, the prediction model is a model for predicting acceptability of a transport request by the facility, that is, a likelihood of the acceptance request being accepted. More specifically, in the embodiment, the generation of the prediction model is a process of calculating a coefficient vector to be applied to the feature vector, which is used for calculation of a score value indicating the acceptability of the transport request by the facility. This process may be started at any timing and, for example, may be started automatically at certain time intervals or may be started using an operation of an operator as a trigger.

In step S301, the control unit 20 reads the performance data D1 stored in the performance data storage unit 31 under control of the prediction model generation data acquisition unit 23.

Similarly, in step SS02, the prediction model generation data acquisition unit 23 reads the past probability data D2 stored in the past probability data storage unit 32. Step S302 may be executed after step S301, may be executed concurrently with step S301, or may be executed before step S301.

In step S303, the prediction model generation data acquisition unit 23 refers to values of specific columns from the performance data D1, extracts the past probability data corresponding to these conditions from the past probability data D2, combines the past probability data with the performance data D1, and acquires the prediction model generation data D3. For example, the prediction model generation data acquisition unit 23 refers to values of a hospital ID column, a day-of-week column, and a clinic practice area column from the performance data D1, extracts past probability data corresponding to those conditions from the past probability data D2, combines the past probability data with the performance data D1, and acquires the prediction model generation data D3.

FIG. 8 illustrates an example of the prediction model generation data D3. The prediction model generation data D3 includes, for example, a hospital ID, an acceptance result, a date and time, a day of the week, weather, a clinic practice area, a complexion of a patient, and a heart rate of the patient extracted from the performance data D1, probabilities in units of years and units of months to which a condition of a day of the week corresponds, which are extracted from the past probability data D2, and a probability calculated in units of years and units of months to which a condition of the clinic practice area corresponds. In order to extract the data from the past probability data D2, the prediction model generation data acquisition unit 23 may refer not only to the day-of-week column and the clinic column, but also to a weather column or other columns of the performance data D1.

In step S304, the learning unit 24 performs statistical analysis on the prediction model generation data D3 acquired from the prediction model generation data acquisition unit 23 and generates the prediction model. In this embodiment, the learning unit 24 executes the statistical analysis in which the acceptance result column (acceptable/not acceptable) in the prediction model generation data D3 is an objective variable and all or some of the other information is explanatory variables (feature vectors). Using this statistical analysis, the learning unit 24 calculates a coefficient vector for calculating a score value indicating acceptability (a level of the likelihood of the acceptance of the acceptance request) of the facility. For example, a case in which the acceptance result column is “acceptable” is labeled as 1, a case in which the acceptance result column is “not acceptable” is labeled as 0, and the learning unit 24 performs analysis using this as an objective variable. When the attribute information associated with each facility, such as the number of beds or information on a specialist, is included in the data D3 as described above, the learning unit 24 can use the attribute information for learning or can use the attribute information for learning in combination with the past probability.

As the statistical analysis executed in the learning unit 24, a scheme such as logistic regression analysis, ranking learning, and random forest, for example, may be selected depending on the purpose. Here, a function f(x;W) that outputs a great scalar value when the transport request is “accepted” is designed for the feature vector. Here, x represents the feature vector, and W represents the coefficient vector corresponding to the feature vector. When the number of variables in the feature vector is large, variable selection may be performed. A stepwise method using Akaike information criterion (AIC), Lasso, or the like can be applied to the variable selection. A final parameter W can be calculated using a Newton-Raphson method, or the like. When the feature vector is category data, a vector subjected to conversion to dummy variables can be set as the feature vector. Further, for example, a case in which the acceptance result column is “acceptable” is labeled as 1, a case in which the acceptance result column is “not acceptable” is labeled as 0, and the analysis is performed using this as an objective variable.

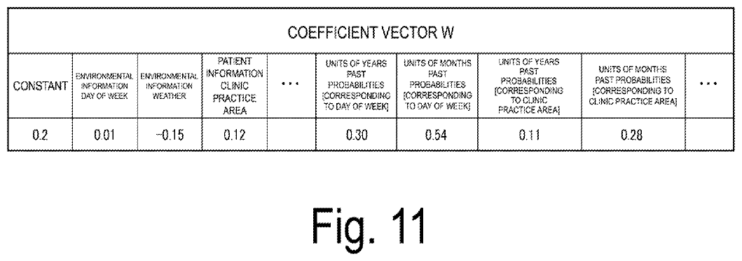

In step S305, the control unit 20 stores the calculated final parameter as a coefficient vector W in the prediction model storage unit 33. FIG. 11 is a diagram illustrating an example of the coefficient vector W. In FIG. 11, for convenience, the coefficient vector W is represented as including a constant term.

### (3) Calculation of Score Value

### (3-1) Acquisition of Score Calculation Data

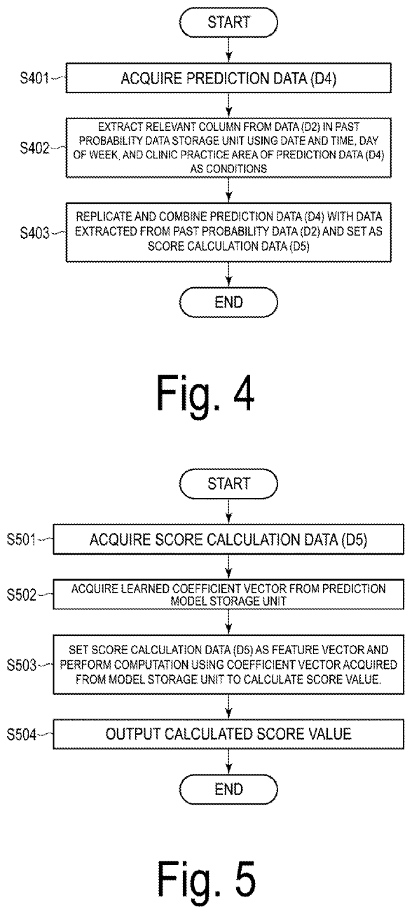

FIG. 4 is a flow diagram illustrating an example of a processing procedure and processing content of a score calculation data acquisition process in the control unit 20 of the selection assistance apparatus 1 illustrated in FIG. 1. This process is started, for example, in response to an input of a start request from a user or an operator (for example, rescue personnel or an operator of a service center) when there is a new patient needing rescue transport.

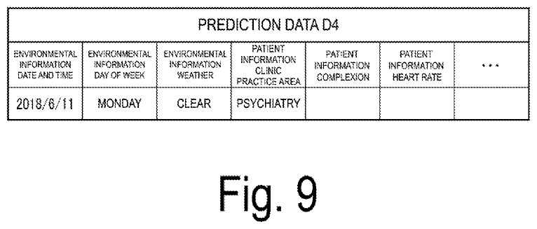

In step S401, the control unit 20 acquires the prediction data D4 for a newly generated request under control of the prediction data acquisition unit 25. FIG. 9 illustrates an example of the prediction data D4. For example, the prediction data D4 includes attribute information relevant to a newly requested rescue transport as the newly generated acceptance request, and more specifically, includes patient information such as a clinic practice area, a complexion, and a heart rate depending on a symptom of a person scheduled to be transported (a patient), in addition to the environmental information such as a date and time, a day of the week, and weather.

In step S402, the control unit 20 sets a specific column of the prediction data D4 as a condition and extracts only the column from the past probability data D2 stored in the past probability data storage unit 32 under control of the past probability data acquisition unit 26. For example, the past probability data acquisition unit 26 sets a day-of-week column and the clinic practice area column of the prediction data D4 as a condition and extracts the column from the past probability data D2.

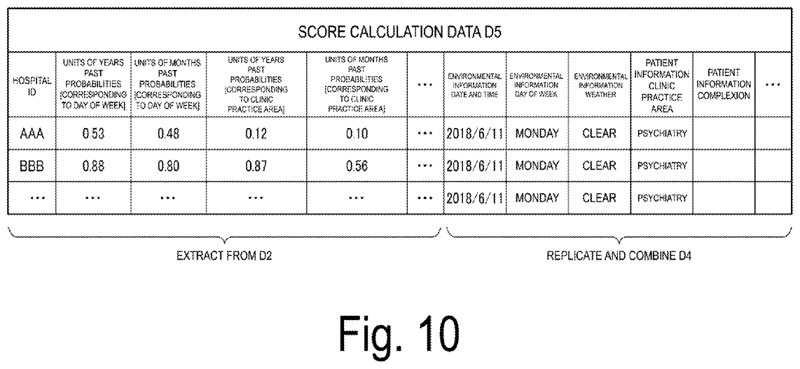

In step S403, the control unit 20 replicates the acquired prediction data D4, combines the replicated data with the data extracted from the past probability data D2, and sets the resultant data as the score calculation data D5 under control of the past probability data acquisition unit 26. Because the number of records of the data extracted from the past probability data D2 corresponds to the number of hospitals, the prediction data D4 corresponding to the number of records of the past probability data D2 is replicated and combined. FIG. 10 illustrates an example of the score calculation data D5. The score calculation data D5 includes a hospital ID, and probabilities in units of years and units of months extracted from the past probability data D2 corresponding to the day of the week and the clinic practice area extracted from the prediction data D4 for each hospital

### (3-2) Score Calculation Process

FIG. 5 is a flow diagram illustrating an example of a processing procedure and processing content of a score calculation process of the control unit 20 of the selection assistance apparatus 1 illustrated in FIG. 1. This process is typically carried out following the process of (3-1) Acquisition of Score Calculation Data.

In step S501, the control unit 20 acquires the score calculation data D5 generated as described above from the past probability data acquisition unit 26 under control of the score calculation unit 27.

In step S502, the score calculation unit 27 acquires the coefficient vector W as a learned prediction model stored in the prediction model storage unit 33.

In step S503, the score calculation unit 27 sets the score calculation data D5 as a feature vector, and performs a computation using the coefficient vector W acquired from the prediction model storage unit 33 to calculate the score value. The score value indicates acceptability of a request regarding each hospital, and a higher score value means that acceptability of the transport request is higher.

Here, the feature vector indicates the same columns as those included in the coefficient vector W, and the score calculation unit 27 does not set columns not included in the coefficient vector W as the feature vector. When the feature vector is the category data, the score calculation unit 27 sets a vector subjected to conversion to dummy variables as the feature vector.

As a method of calculating the score value,

Score value=1/(1+exp(−(t(W)X)))

can be calculated because a value of a function f(x;W) obtained by the learning unit 24 is expressed as t(W)X. Here, t denotes a transposition.

In step S504, the control unit 20 performs a process of outputting the score value calculated by the score calculation unit 27 under control of the output control unit 28. For example, the output control unit 28 can sort the calculated score values in decreasing order to create, as output data, a priority list obtained by assigning a priority to a plurality of hospitals that are transport destination candidates. Here, the output control unit 28 may create the calculated score values as the output data as is, or may create the score values as the output data in which candidate facilities other than the sorted upper-ranked candidate facilities are excluded. Further, when a distance between a place at which a patient appears and each hospital is known in advance, a threshold value may be set for the distance to narrow down hospitals to be displayed, or the distance may be displayed as a set with the score value.

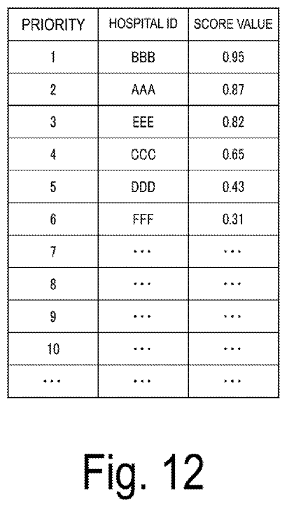

FIG. 12 illustrates an example of output data including the calculated score value. In FIG. 12, a list of priorities sorted in descending order from a priority with a higher score value to a priority with a lower score value based on the calculated score values is illustrated as output data. A higher score of the hospital indicates a high likelihood of the transport request being accepted. Thus, the priority list in FIG. 12 indicates that hospital BBB having the highest score value 0.95 has the highest likelihood of acceptance of the transport request, hospital AAA (score value 0.87) has the second highest likelihood of acceptance of the transport request, and hospital EEE (score value 0.82) has the third highest likelihood of acceptance of the transport request. By setting this priority list as the output data, a user or operator viewing the priority list can immediately determine that hospital BBB has a high likelihood of a current transport request being accepted and outputs the transport request to hospital BBB. Even when the acceptance is rejected by the hospital BBB, the second hospital AAA can be selected immediately as a next candidate, and thus the user or operator viewing the priority list can minimize the time required for selection of a transport destination candidate. To enhance convenience for the user, the output control unit 28 may also output a facility name instead of the hospital ID.

### Second Example

A second example of the present invention is an example in which a learning model is generated for each clinic practice area. Therefore, in the second example, data divided for the clinic practice area is used as the prediction model generation data D3.

Operations of the second example will also be described with reference to FIGS. 2 to 5 as in the first example, but the same operations as those of the first example will be omitted.

(1) Calculation of Past Probability

The past probability calculation process can be started at any timing, as in the first example.

In step S201 of FIG. 2, the control unit 20 acquires the performance data D1 according to the past transport performance from an input device, an external database, or the like via the input and output interface unit 10 and stores the performance data D1 in the performance data storage unit 31 under control of the performance data acquisition unit 21. FIG. 6 illustrates an example of the acquired performance data D1.

In step S202, the control unit 20 performs a process of reading the performance data D1 from the performance data storage unit 31, referring to the column of the hospital ID of the performance data D1, creating a unique list of hospital IDs, and dividing the performance data D1 for each hospital ID under control of the past probability calculation unit 22.

Subsequently, in step S203, the past probability calculation unit 22 extracts data in units of years for each piece of data divided for each hospital ID.

Then, in step S204, the past probability calculation unit 22 calculates, for each hospital, past probabilities for each clinic practice area and each day of the week based on the data extracted in units of years. The past probability is calculated to range from 0 to 1, as in the first example.

In step S205, the past probability calculation unit 22 extracts data in units of months for each piece of data divided for each hospital ID.

Then, in step S206, the past probability calculation unit 22 calculates the past probabilities for each clinic practice area and each day of the week based on the data extracted in units of months.

In step S207, the past probability calculation unit 22 combines the calculated past probabilities with a corresponding hospital ID in the unique list of the hospital IDs and sets the resultant data as the past probability data D2. FIG. 7 illustrates an example of the past probability data D2.

In step S208, the past probability calculation unit 22 stores the acquired past probability data D2 in the past probability data storage unit 32.

(2) Generation of Prediction Model (Calculation of Coefficient Vector)

The process of generating the prediction model can be started at any timing, as in the first example.

In step S301 of FIG. 3, the control unit 20 reads the performance data D1 stored in the performance data storage unit 31 under control of the prediction model generation data acquisition unit 23.

Similarly, in step SS02, the prediction model generation data acquisition unit 23 reads the past probability data D2 stored in the past probability data storage unit 32. Step S302 may be executed after step S301, may be executed concurrently with step S301, or may be executed before step S301.

Then, in step S303, the prediction model generation data acquisition unit 23 refers to values of specific columns from the performance data D1, extracts the past probability data corresponding to these conditions from the past probability data D2, combines the past probability data with the values of specific columns, and acquires the prediction model generation data D3. For example, the prediction model generation data acquisition unit 23 refers to the values of the hospital ID column, the day-of-week column, and the clinic practice area column from the performance data D1, extracts the past probability data corresponding to those conditions from the past probability data D2, combines the past probability data with the performance data D1, and acquires the prediction model generation data D3.

Here, in the second example, the prediction model generation data acquisition unit 23 generates the prediction model generation data D3 divided into the data for each clinic practice area in order to create a learning model for each clinic practice area, unlike the first example.

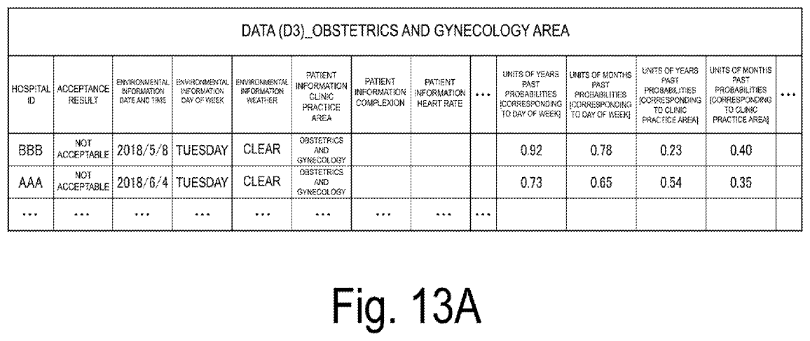

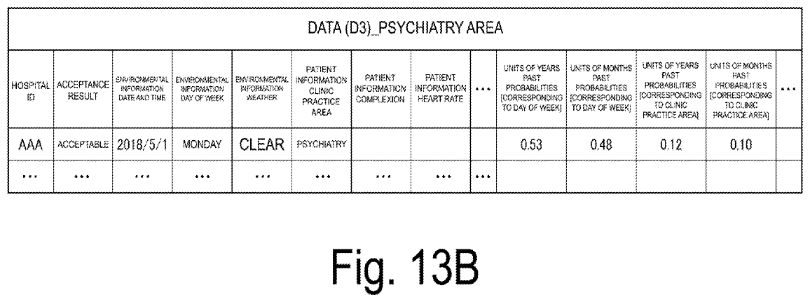

FIG. 13A illustrates data corresponding to obstetrics and gynecology area among the data divided for each clinic practice area as a second example of the prediction model generation data D3. FIG. 13B illustrates data corresponding to a psychiatry area among the data divided for each clinic practice area as a third example of the prediction model generation data D3.

In step S304 of FIG. 3, in this example, the learning unit 24 executes the statistical analysis in which the acceptance result column in the prediction model generation data D3 is an objective variable and all or some of the other information is explanatory variables (feature vectors). Using this statistical analysis, the learning unit 24 calculates the coefficient vector W for calculating a score value indicating the acceptability. The calculation of the coefficient vector W may employ the same operations as those of the first example, and thus detailed description thereof will be omitted.

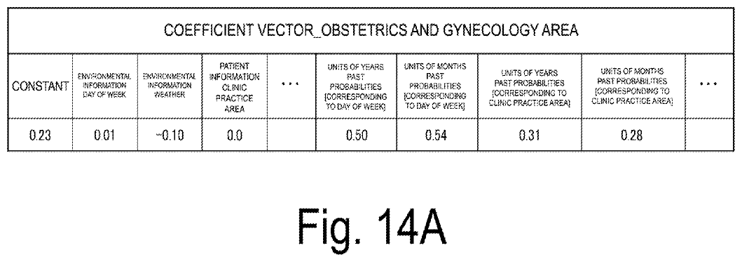

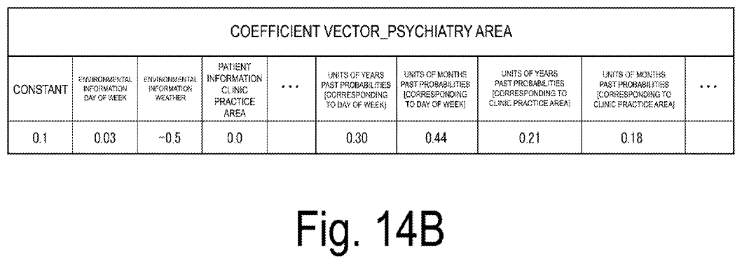

Through the above process, the coefficient vector W is calculated for each clinic practice area. FIG. 14A illustrates a coefficient vector calculated for each clinic practice area corresponding to the obstetrics and gynecology area as a second example of the coefficient vector W, and FIG. 14B illustrates a coefficient vector calculated for each clinic practice area corresponding to a psychiatry area as a third example of the coefficient vector W.

(3) Calculation of Score Value

### (3-1) Acquisition of Score Calculation Data

A process of acquiring the score calculation data is started, for example, in response to an input of a start request from a user or an operator (for example, rescue personnel or an operator of a service center) when there is a new patient needing rescue transport, as in the first example.

In step S401 of FIG. 4, the control unit 20 acquires the prediction data D4 for a newly generated request under the control of the prediction data acquisition unit 25. FIG. 9 illustrates an example of the prediction data D4.

In step S402, the control unit 20 sets a specific column of the prediction data D4 as a condition and extracts only the column from the past probability data D2 stored in the past probability data storage unit 32 under control of the past probability data acquisition unit 26.

In step S403, the control unit 20 replicates the acquired prediction data D4, combines the replicated data with the data extracted from the past probability data D2, and sets the resultant data as the score calculation data D5 under control of the past probability data acquisition unit 26. Because the number of records of the data extracted from the past probability data D2 corresponds to the number of hospitals, the prediction data D4 corresponding to the number of records of the past probability data D2 is replicated and combined. FIG. 10 illustrates an example of the score calculation data D5.

### (3-2) Score Calculation Process

A score calculation process is typically executed following the process of (3-1) Acquisition of Score Calculation Data, as in the first example.

In step S501 of FIG. 5, the control unit 20 acquires the score calculation data D5 generated by the past probability data acquisition unit 26 as described above under the control of the score calculation unit 27.

In step S502, the score calculation unit 27 acquires the coefficient vector W as the learned prediction model stored in the prediction model storage unit 33. In a second example, because the coefficient vector is calculated for each clinic practice area as described above, the score calculation unit 27 refers to a clinic practice area column of patient information in the score calculation data D5 and selects a relevant coefficient vector from the prediction model storage unit 33. In the example illustrated in FIG. 10, because the clinic practice area column of the score calculation data D5 indicates a psychiatry area, the score calculation unit 27 reads the coefficient vector for each clinic practice area corresponding to the psychiatry area illustrated in FIG. 14B.

In step S503, the score calculation unit 27 sets the score calculation data D5 as a feature vector and performs a computation using the coefficient vector W for each clinic practice area acquired from the prediction model storage unit 33 to calculate the score value. The score value indicates acceptability of a request regarding each hospital, and a higher score value means that acceptability of the transport request is higher.

Here, the feature vector indicates the same columns as those included in the coefficient vector W. and the score calculation unit 27 does not set columns not included in the coefficient vector W as the feature vector. When the feature vector is the category data, the score calculation unit 27 sets a vector subjected to conversion to dummy variables as the feature vector. For the method of calculating the score value, the same method as in the first example may be employed.

In step S504, the control unit 20 performs a process of outputting the score value calculated by the score calculation unit 27 under control of the output control unit 28. Even when the coefficient vector W for each clinic practice area has been used, the score value is calculated for each hospital, as in the first example.

Verification

Validation was performed using performance data from January to December of 2017 in order to evaluate the validity of the score values calculated according to the embodiment. 80 percent of the overall performance data was used as learning data, and the remaining 20 percent was used as verification data.

A value of an area under the curve (AUC) based on a receiver operating characteristic (ROC) curve was used as an evaluation index. The AUC value is an evaluation index based on the ROC curve that is typically used often to indicate accuracy of binary classification. Because a discrimination capacity is high when the AUC value is higher, content is correctly ranked according to a score in order from a positive example to a negative example when the AUC value is used as the evaluation index. When the discrimination capacity is random, the AUC value is 0.5.

More specifically, the AUC value is calculated using the following equation:

$$\begin{matrix}\overline{AUC} = \frac{1}{{N}^{+} {N}^{-}} {\sum }_{i = 1}^{{N}^{+}} {\sum }_{j = 1}^{{N}^{-}} I ( f ( {x}_{i}^{+} \text{;} W ) > f ( {x}_{j}^{-} \text{;} W ) ) \\ Here , & [ Math . 1 ] \\ I ( f ( {x}_{i}^{+} \text{;} W ) > f ( {x}_{j}^{-} \text{;} W ) ) & [ Math . 2 ]\end{matrix}$$

is a step function of outputting 1 when

f(xᵢ⁺;W)>f(xᵢ⁻;W) [Math. 3]

and otherwise outputting 0.

According to the first example, the coefficient vector for calculating the score value of the acceptability indicating that a certain hospital is acceptable using learning data was obtained using the selection assistance apparatus 1 according to the embodiment. Using the coefficient vector and verification data, the score value was calculated by the score calculation unit 27, and the AUC value was calculated for evaluation of accuracy of the score value. As a result, the AUC value was calculated as 0.82.

According to the second example, the coefficient vector for calculating the score value of the acceptability indicating that a certain hospital is acceptable when a request for transport to a psychiatry and neurology area due to a symptom of a patient has been made using the learning data was obtained using the selection assistance apparatus 1 according to the embodiment. Using the coefficient vector and verification data, the score value was calculated by the score calculation unit 27, and the AUC value was calculated for evaluation of accuracy of the score value. As a result, the AUC value was calculated as 0.97.

When hospitals are sorted randomly without using the selection assistance apparatus 1, the AUC value is 0.5.

It is shown that, with the selection assistance apparatus 1, the AUC value can be improved to 0.82 in the first example and the AUC value can be improved to 0.97 in the second example, and thus the score value obtained using the selection assistance apparatus 1 is effective for prediction of the acceptability.

That is, it is shown that, when there are a plurality of hospital candidates that make the transport request, the score value of the acceptability is calculated using a condition of a patient, a past probability of each hospital, or the like, and a sorting order of the priority list created by sorting the score values in descending order is obtained with high accuracy by using the selection assistance apparatus 1 according to the embodiment.

### Effects of the Invention

In the embodiment, the selection assistance apparatus 1 acquires the performance data D1 in which information indicating whether or not the transport request is accepted at each facility is associated with the attribute information (or the features extracted from the attribute information) relevant to the transport request, as described in detail above. Further, the selection assistance apparatus 1 calculates, for each facility, the past probability (past probability data D2) depending on each piece of attribute information (or feature) based on the performance data D1. Further, the selection assistance apparatus 1 combines the performance data D1 with the past probability extracted from the past probability data D2 based on the attribute information (or features) to generate the prediction model generation data D3. Using the prediction model generation data D3, the selection assistance apparatus 1 generates the learned model through statistical analysis in which information indicating whether or not the transport request is accepted is an objective variable and at least one of the attribute information (or features) and the calculated past probability is an explanatory variable.

The learning model generated in this way is a highly reliable model based on past statistical data and is a highly accurate model considering the attribute information. Thus, when a new acceptance request is generated, the selection assistance apparatus 1 can predict, with high accuracy, the likelihood of the acceptance request being accepted for each candidate facility using the generated learned model based on the attribute information (or features) relevant to the acceptance request.

Further, when anew acceptance request is generated, the selection assistance apparatus 1 acquires the attribute information relevant to the acceptance request as the prediction data D4, extracts relevant past probability data from the past probability data D2 based on the attribute information included in the prediction data D4, and combines the extracted past probability data with the prediction data D4 to acquire the score calculation data D5.

Using this score calculation data D5 and the generated learned model (coefficient vector), the selection assistance apparatus 1 calculates a score value indicating the level of the likelihood of the acceptance of the acceptance request for each candidate facility. The calculated score value is output together with information for identifying the candidate facility as a prediction result.

Thus, the prediction result is output as the score value, thereby facilitating a process of calculating the prediction result. For example, the prediction result can be utilized in various ways, such as sorting in descending order of the score values, comparing the score values to a predetermined threshold value, or labeling through classification. Further, it is possible to curb a processing load of the apparatus by selecting an output of the prediction result depending on the score value.

The user or operator can find the candidate facility having a high score value from the output result to immediately identify the facility that can easily accept the transport request. This allows the user or operator to preferentially output the transport request to a hospital having a higher score value and efficiently perform selection of and transport to the candidate facility when there is a patient needing transport. Further, even when acceptance is rejected, the user or operator can select a hospital having a second highest score value to select the next facility as a transport request destination immediately, and thus it is possible to minimize a required transport time.

In the embodiment, because a facility having a high likelihood of acceptance can be easily determined based on the output score value, the user or operator does not need to further identify a request transmission destination from among the plurality of candidate facilities. Further, in the embodiment, because the selection assistance apparatus 1 does not use hospital visit record of a specific patient as past statistical data, a candidate facility is not unnecessarily limited. This allows the selection assistance apparatus 1 to recommend a hospital having a high likelihood of acceptance based on the attribute information of the patient even when the hospital has no newly generated hospital visit history of the patient, and to find a hospital having a higher likelihood of acceptance from among more candidate facilities. Further, in the embodiment, a rescue vehicle and each hospital need not be connected to the communication network in advance. Further, the processes according to the embodiment do not require complex operations by the rescue personnel or operator performing the transport request.

Thus, the selection assistance apparatus 1 can efficiently perform the selection of the candidate facility and minimize a time required until the acceptance destination is determined, and achieve reduction in a working burden on a user making a request, such as rescue personnel or an operator performing rescue transport. Further, a patient to be transported is able to undergo rapid treatment, such that the selection assistance apparatus 1 can reduce a burden on the transported person.

Further, the selection assistance apparatus 1 considers various features extracted from the attribute information of the acceptance request to calculate past probability data, for example, for each clinic practice area and use the past probability data for analysis, thereby generating a more precise learning model satisfying detailed conditions. This allows the selection assistance apparatus 1 to perform high accuracy prediction using a learning model generated under the detailed conditions further satisfying transport conditions w % ben a patient needing transport newly appears.

### OTHER EMBODIMENTS

The disclosure is not limited to the above-described embodiment. For example, the configuration of each unit included in the control unit 20, the configuration of the record stored in the storage unit, and the like can be implemented with various modifications without departing from the gist of the present invention.

Further, a case in which an example of the request from the user is a request for rescue transport has been described, but the present invention is not limited to this case. The embodiments are applicable to cases in which a rapid response other than rescue transport is desired, such as various cases in which an acceptance request needs to be output to a facility, for example, for selection of a hospital change destination when a hospital change is required due to a sudden change in a symptom of a patient, and securing of a temporary accommodation destination of a victim at the time of occurrence of disaster. A facility that is an acceptance destination candidate is also not limited to a medical institution. For example, the above embodiments are also applicable to a case in which a variety of facilities likely to reject acceptance when an acceptance request is made, such as nursing facilities, educational facilities, lodging facilities, amusement facilities, sports facilities, conference rooms, theaters, and event venues are selected.

Further, a wide variety of information can be adopted as attribute information (features) or conditions relevant to the request. For example, when there is a request for rescue transport as the acceptance request, various types of information such as information of a time period such as early morning/daytime/night/morning/afternoon, information in units of days such as weekdays/holidays/public holidays, weather, temperature, and humidity can be used as the environmental information. Similarly, a variety of information such as a sex, an age, a degree of bleeding, and a level of consciousness of a patient can be used as the patient information. In the case of an acceptance request of things other than the rescue transport, a wide variety of information such as a purpose, a capacity of people, the presence or absence of qualified persons, acoustic equipment, and a budget of an event can be assumed as other attribute information. Among such a wide variety of attribute information, attribute information that is adopted as data extraction conditions may be set according to predetermined criteria in advance or may be selected appropriately by an operator. Further improvement of prediction accuracy is expected by selecting optimal conditions depending on a purpose of the request.

Further, the selection assistance apparatus 1 may be an apparatus that can be directly operated for an input by rescue personnel or may be a server disposed on a cloud. For example, when the selection assistance apparatus 1 is the server and the rescue personnel inputs information on a patient that is a transport target through a terminal of the rescue personnel, the selection assistance apparatus 1 can be configured to receive the input patient information via a wireless network. The selection assistance apparatus 1 may transmit the priority list including the score values calculated by executing the various processes to the terminal of the rescue personnel via the wireless network so that the priority list is displayed on a display of the terminal of the rescue personnel.

Further, an example in which the priority list is output based on the score values calculated for each candidate facility has been described, but an output format is not limited thereto. For example, only an upper-ranked candidate facility name may be output instead of the score value, or a facility of which the likelihood of acceptance is determined to satisfy a predetermined criterion may be displayed in a different color on a map.

Further, a data structure of the data D1 to D5, for example, can be variously modified and implemented without departing from the gist of the present invention. For example, the selection assistance apparatus 1 can use data in any period of time including any point in time to generate the data set D2 indicating the probability that the past request was accepted. The selection assistance apparatus 1 can use the various attribute information (or features) described above alone or in any combination for learning or for calculation of a probability for learning (a past probability of acceptance). For example, in the example, the selection assistance apparatus 1 extracts the data for probability calculation using each of the clinic practice area and the day of the week as a single condition, but it may extract the data using any combination condition, such as a combination of the clinic practice area and the day of the week or a combination of the clinic practice area, the day of the week, and the weather.

In short, the disclosure is not limited to the above-described embodiment as it is, and can be embodied with the components modified without departing from the scope of the disclosure when implemented. Furthermore, various inventions can be formed by appropriate combinations of a plurality of components disclosed in the above-described embodiment. For example, several components may be deleted from all of the components illustrated in the embodiment. Furthermore, components of different embodiments may be appropriately combined with each other.

### REFERENCE SIGNS LIST

1 Selection assistance apparatus
10 Input and output interface unit
20 Control unit
21 Performance data acquisition unit
22 Past probability calculation unit
23 Prediction model generation data acquisition unit
24 Learning unit
25 Prediction data acquisition unit
26 Past probability data acquisition unit
27 Score calculation unit
28 Output control unit
30 Storage unit
31 Performance data storage unit
32 Past probability data storage unit
33 Prediction model storage unit

1. A selection assistance apparatus for assisting in selecting an acceptance destination facility in response to a request from a user, the selection assistance apparatus comprising:
an acceptance performance data acquisition unit including one or more processors, configured to acquire acceptance performance data in which information indicating success or failure of acceptance for a past acceptance request in each of a plurality of candidate facilities is associated with attribute information relevant to the past acceptance request;
a past probability calculation unit, including one or more processors, configured to calculate a past probability of acceptance according to the attribute information in each of the plurality of candidate facilities based on the acquired acceptance performance data; and
a learning unit, including one or more processors, configured to generate a prediction model for predicting a likelihood of acceptance for a newly generated acceptance request according to attribute information relevant to the newly generated acceptance request for each of the plurality of candidate facilities based on the acceptance performance data and the calculated past probability, the prediction model indicating a relationship between information indicating success or failure of the acceptance and the attribute information.

2. The selection assistance apparatus according to claim 1, further comprising:
an acceptance likelihood prediction unit including one or more processors, configured to predict a likelihood of acceptance of the newly generated acceptance request based on the generated prediction model and attribute information relevant to the newly generated acceptance request for each of the plurality of candidate facilities; and
an output unit including one or more processors, configured to output a result of the prediction of the acceptance likelihood prediction unit.

3. The selection assistance apparatus according to claim 2,
wherein the acceptance likelihood prediction unit further calculates a score value indicating a level of the likelihood of acceptance; and
the output unit sorts and outputs the calculated score values.

4. The selection assistance apparatus according to claim 1, wherein the learning unit generates the prediction model for each feature type focusing on at least one of a plurality of features extracted from the attribute information.

5. The selection assistance apparatus according to claim 1,
wherein the past probability calculation unit calculates a past probability in each of the plurality of candidate facilities under conditions corresponding to each of a plurality of features extracted from the attribute information relevant to the past acceptance request, and
the learning unit generates the prediction model using information indicating success or failure of the acceptance as an objective variable, and at least one of the plurality of features and the past probability as an explanatory variable.

6. A selection assistance method executed by a selection assistance apparatus for assisting in selecting an acceptance destination facility in response to a request from a user, the selection assistance method comprising:
acquiring acceptance performance data in which information indicating success or failure of acceptance for a past acceptance request in each of a plurality of candidate facilities is associated with attribute information relevant to the past acceptance request;
calculating a past probability of acceptance according to the attribute information in each of the plurality of candidate facilities based on the acquired acceptance performance data; and
generating a prediction model for predicting a likelihood of acceptance for a newly generated acceptance request according to attribute information relevant to the newly generated acceptance request for each of the plurality of candidate facilities based on the acceptance performance data and the calculated past probability, the prediction model indicating a relationship between information indicating success or failure of the acceptance and the attribute information.

7. (canceled)

8. (canceled)

9. (canceled)

10. A non-transitory computer readable medium storing one or more instructions causing a processor to execute:
acquiring acceptance performance data in which information indicating success or failure of acceptance for a past acceptance request in each of a plurality of candidate facilities is associated with attribute information relevant to the past acceptance request;
calculating a past probability of acceptance according to the attribute information in each of the plurality of candidate facilities based on the acquired acceptance performance data; and
generating a prediction model for predicting a likelihood of acceptance for a newly generated acceptance request according to attribute information relevant to the newly generated acceptance request for each of the plurality of candidate facilities based on the acceptance performance data and the calculated past probability, the prediction model indicating a relationship between information indicating success or failure of the acceptance and the attribute information.

11. The selection assistance method according to claim 6, further comprising:
predicting a likelihood of acceptance of the newly generated acceptance request based on the generated prediction model and attribute information relevant to the newly generated acceptance request for each of the plurality of candidate facilities; and
outputting a result of the prediction.

12. The selection assistance method according to claim 11, further comprising:
calculating a score value indicating a level of the likelihood of acceptance; and
sorting and outputting the calculated score values.

13. The selection assistance method according to claim 6, further comprising:
generating the prediction model for each feature type focusing on at least one of a plurality of features extracted from the attribute information.

14. The selection assistance method according to claim 6, further comprising:
calculating a past probability in each of the plurality of candidate facilities under conditions corresponding to each of a plurality of features extracted from the attribute information relevant to the past acceptance request, and
generating the prediction model using information indicating success or failure of the acceptance as an objective variable, and at least one of the plurality of features and the past probability as an explanatory variable.

15. The non-transitory computer readable medium according to claim 10, wherein the one or more instructions further cause the processor to execute:
predicting a likelihood of acceptance of the newly generated acceptance request based on the generated prediction model and attribute information relevant to the newly generated acceptance request for each of the plurality of candidate facilities; and
outputting a result of the prediction.

16. The non-transitory computer readable medium according to claim 15, wherein the one or more instructions further cause the processor to execute:
calculating a score value indicating a level of the likelihood of acceptance; and
sorting and outputting the calculated score values.

17. The non-transitory computer readable medium according to claim 10, wherein the one or more instructions further cause the processor to execute:
generating the prediction model for each feature type focusing on at least one of a plurality of features extracted from the attribute information.

18. The non-transitory computer readable medium according to claim 10, wherein the one or more instructions further cause the processor to execute:
calculating a past probability in each of the plurality of candidate facilities under conditions corresponding to each of a plurality of features extracted from the attribute information relevant to the past acceptance request, and
generating the prediction model using information indicating success or failure of the acceptance as an objective variable, and at least one of the plurality of features and the past probability as an explanatory variable.
